(19) United States
(12) Patent Application Publication
(10) Pub. No.: US 2023/0115453 A1
(43) Pub. Date: Apr. 13, 2023

(54) ELECTROOSMOTIC PUMP, METHOD FOR MANUFACTURING SAME, AND FLUID PUMPING SYSTEM COMPRISING SAME

(71) Applicant: EOFLOW CO., LTD., Seongnam-si, Gyeonggi-do (KR)

(72) Inventors: Young Wook Chang, Seoul (KR); Jae Hong Kim, Seongnam-si, Gyeonggi-do (KR); Yongchul Song, Seongnam-si, Gyeonggi-do (KR); Mihyun Lee, Seongnam-si, Gyeonggi-do (KR)

(21) Appl. No.: 17/906,185

(22) PCT Filed: Mar. 12, 2021
(86) PCT No.: PCT/KR2021/003121

§ 371 Date: Sep. 12, 2022

(30) Foreign Application Priority Data
Mar. 13, 2020 (KR) 10-2020-0031183

(51) Int. Cl. F04B 19/00

(52) U.S. Cl. CPC F04B 19/006; A61M 5/14244

(57) ABSTRACT

Provided are an electroosmotic pump including a membrane; a first electrode disposed on a surface of the membrane and including a first porous support including a first conductor and a first precious metal and a first conductive polymer positioned in or on at least some of pores and surfaces of the first porous support; and a second electrode disposed on another surface of the membrane and including a second porous support including a second conductor and a second precious metal and a second conductive polymer positioned in or on at least some of pores and surfaces of the second porous support, a method of manufacturing the electroosmotic pump, and a fluid pumping system including the electroosmotic pump.

The present disclosure relates to an electroosmotic pump, a method of manufacturing the same, and a fluid pumping system including the same.

### BACKGROUND ART

An electroosmotic pump is a pump using the movement of a fluid that occurs when a voltage is applied to both ends of a capillary tube or a porous membrane. Therefore, in the case of configuring a pump using electroosmosis, it is essential to use an electrode for applying a voltage to both ends of a capillary tube or a porous membrane.

In general, chemically stable platinum has been used as a material for an electrode. However, since platinum exhibits a low hydrogen overpotential to water, when a potential difference of several V or greater is applied to both ends of a porous membrane, hydrogen gas is generated at a reduction electrode, and such gas generation is a factor limiting the practical use of an electroosmotic pump.

In addition to suppression of gas generation at a reduction electrode, it is necessary to generate a sufficient pressure and a sufficient flow rate for the practical feasibility of an electroosmotic pump.

Also, the amount of power required to drive a pump is an important factor determining practical feasibility when applied as a patch-type drug delivery device or a wearable medical device to be attached to a human body.

As a method of delivering drugs by implanting a small pump inside the human body is being spotlighted, interest in an electroosmotic pump that may be operated stably is increasing, and thus research is being conducted to improve the stability, the lifespan characteristics, and the efficiency of an electroosmotic pump.

### DESCRIPTION OF EMBODIMENTS

### Technical Problem

The present disclosure provides an electroosmotic pump, which is capable of minimizing gas generation at an electrode, may be operated normally even when a gas is generated, and has a wide driving voltage range to control a pressure and a flow rate.

The present disclosure also provides a method of manufacturing the electroosmotic pump.

The present disclosure also provides a fluid pumping system including the electroosmotic pump.

### Technical Solution to Problem

According to an embodiment of the present disclosure, an electroosmotic pump includes a membrane; an electrode A provided on a surface of the membrane; and an electrode B provided on another surface of the membrane, wherein at least one of the electrode A and the electrode B includes a porous support including a conductor and a precious metal and a conductive polymer positioned in or on at least some of pores and surfaces of the porous support.

Unless described otherwise below, descriptions of the electrode A, the electrode B, the conductor, the porous support, the precious metal, and the conductive polymer may be identical or corresponding to the descriptions of a first electrode, a second electrode, a first conductor, a second conductor, a first porous support, a second porous support, a first precious metal, a second precious metal, a first conductive polymer, and a second conductive polymer described below.

One of the electrode A and the electrode B not having a structure including a porous support including a conductor and a precious metal and a conductive polymer located in or on at least some of pores and surfaces of the porous support may have, but is not limited to, a generally used porous electrode structure including, for example, a structure including a porous conductor and/or an electrochemical reaction material; or a structure including a porous support including a non-conductor and a conductor and/or an electrochemical reaction material formed on the porous support.

According to an embodiment of the present disclosure, an electroosmotic pump includes a membrane; a first electrode disposed on a surface of the membrane and including a first porous support including a first conductor and a first precious metal and a first conductive polymer positioned in or on at least some of pores and surfaces of the first porous support; and a second electrode disposed on another surface of the membrane and including a second porous support including a second conductor and a second precious metal and a second conductive polymer positioned in or on at least some of pores and surfaces of the second porous support.

The membrane may include one or more selected from the group consisting of porous ceramics, anionic organic polymers, cationic organic polymers, and combinations thereof, each of which does not exhibit conductivity.

The first conductor and the second conductor may be the same as or different from each other and each independently include at least one selected from the group consisting of carbon, precious metals, conductive metals, conductive polymers, and combinations thereof.

The first porous support and the second porous support may be the same as or different from each other and each independently have a pore size from about 0.1 μm to about 500 μm.

The first porous support and the second porous support may be the same as or different from each other and each independently have a porosity from about 5% to about 95%.

The first precious metal and the second precious metal may be the same as or different from each other and may each independently include one or more selected from the group consisting of platinum, gold, silver, palladium, iridium, and combinations thereof.

The first precious metal and the second precious metal may be the same as or different from each other and each independently have an average particle diameter from about 1 nm to about 1 μm.

The first precious metal and the second precious metal may be the same as or different from each other and may each independently be adsorbed or impregnated on a carbon catalyst.

The first conductive polymer and the second conductive polymer may be the same as or different from each other and each independently include one or more selected from the group consisting of poly(3,4-ethylene dioxythiophene): poly (styrene sulfonate) [PEDOT: PSS], poly(aniline):poly(styrene sulfonate), poly(aniline):camphorsulfonic acid (PANI:CSA), poly(thiophene):poly(styrene sulfonate), polyaniline, polypyrrole, polythiophene, polythionine, quinone-based polymer, and combinations thereof.

The first conductive polymer and the second conductive polymer may be the same as or different from each other and each independently have an average particle diameter from about 1 nm to about 1 μm.

A weight ratio of the first precious metal and the first conductive polymer may be from 1:99 to 30:70. A weight ratio of the second precious metal and the second conductive polymer may be from 1:99 to 30:70. The weight ratio of the first precious metal and the first conductive polymer and the weight ratio of the second precious metal and the second conductive polymer may be the same as or different from each other.

The first precious metal and the first conductive polymer may be present in a form of a first thin-film on at least some of the pores and the surfaces of the first porous support. The second precious metal and the second conductive polymer may be present in a form of a second thin-film on at least some of the pores and the surfaces of the second porous support.

The first thin-film and the second thin-film may be the same as or different from each other and each independently have a thickness from about 1 nm to about 2 μm.

A driving voltage of the electroosmotic pump may be from about 0.1 V to about 5 V.

According to another aspect of the present disclosure, a method of manufacturing an electroosmotic pump according to another embodiment of the present disclosure includes preparing a membrane; fabricating a first electrode by forming a first precious metal and a first conductive polymer positioned in or on at least some of pores and surfaces of a first porous support including a first conductor; fabricating a second electrode by forming a second precious metal and a second conductive polymer positioned in or on at least some of pores and surfaces of a second porous support including a second conductor; and forming a membrane-electrode assembly by bringing the first electrode into contact with a surface of the membrane and brining the second electrode into contact with another surface of the membrane.

The fabricating of the first electrode may include preparing a first dispersed solution including the first precious metal, the first conductive polymer, and a first solvent and deep-coating or spray-coating the first dispersed solution on the first porous support including the first conductor.

The fabricating of the second electrode may include preparing a second dispersed solution including the second precious metal, the second conductive polymer, and a second solvent and deep-coating or spray-coating the second dispersed solution on the second porous support including the second conductor.

According to another aspect of the present disclosure, a fluid pumping system including the electroosmotic pump is provided.

### Advantageous Effects of Disclosure

An electroosmotic pump according to an embodiment may minimize gas generation at an electrode, may be operated normally even when a gas is generated, and has a wide driving voltage range to control a pressure and a flow rate.

### BRIEF DESCRIPTION OF DRAWINGS

FIG. 1 is a configuration diagram schematically showing an electroosmotic pump according to an embodiment of the present disclosure.

FIGS. 2A and 2B are conceptual diagrams schematically showing a reversible electrode reaction in an electroosmotic pump according to an embodiment of the present disclosure and movement of ions and a fluid resulting therefrom.

FIG. 3 is an exploded perspective view of an electroosmotic pump according to an embodiment of the present disclosure.

FIG. 4 is an electron micrographic image of an electrode prepared according to Example 1.

FIG. 5 is a graph of pressures generated by an electroosmotic pump manufactured according to Example 2.

FIG. 6 is a graph of flow rates generated by an electroosmotic pump manufactured according to Example 2.

FIG. 7 is a current characteristic graph obtained by alternately operating an electroosmotic pump manufactured according to Example 2 at 2.5 V and −2.5 V for 4 days or longer.

FIG. 8 is a current characteristic graph obtained by alternately operating an electroosmotic pump manufactured according to Example 2 at 4 V and −4 V for 5500 cycles or more.

### MODE OF DISCLOSURE

Hereinafter, exemplary embodiments of the present disclosure will be described in detail with reference to the accompanying drawings. The disclosure may, however, be embodied in many different forms and should not be construed as limited to the embodiments set forth herein.

In order to clearly express various layers and regions in the drawings, the thicknesses thereof are enlarged. Throughout the specification, like reference numerals denote like elements. When an element is said to be on “one surface”, “on”, or “on a surface” of another element, this includes not only when the element is “immediately adjacent” to the other element, but also the case where another element is present therebetween.

In addition, unless explicitly described to the contrary, the word “comprise” and variations such as “comprises” or “comprising” will be understood to imply the inclusion of stated elements but not the exclusion of any other elements, and a “combination” indicates a mixture, an alloy, a polymerization, or a copolymerization.

According to an embodiment of the present disclosure, an electroosmotic pump includes a membrane; an electrode A provided on a surface of the membrane; and an electrode B provided on another surface of the membrane, wherein at least one of the electrode A and the electrode B includes a porous support including a conductor and a precious metal and a conductive polymer positioned in or on at least some of pores and surfaces of the porous support.

Unless described otherwise below, descriptions of the electrode A, the electrode B, the conductor, the porous support, the precious metal, and the conductive polymer may be identical or corresponding to the descriptions of a first electrode, a second electrode, a first conductor, a second conductor, a first porous support, a second porous support, a first precious metal, a second precious metal, a first conductive polymer, and a second conductive polymer described below.

One of the electrode A and the electrode B not having a structure including a porous support including a conductor and a precious metal and a conductive polymer located in or on at least some of pores and surfaces of the porous support may have, but is not limited to, a generally used porous electrode structure including, for example, a structure including a porous conductor and/or an electrochemical reaction material; or a structure including a porous support including a non-conductor and a conductor and/or an electrochemical reaction material formed on the porous support.

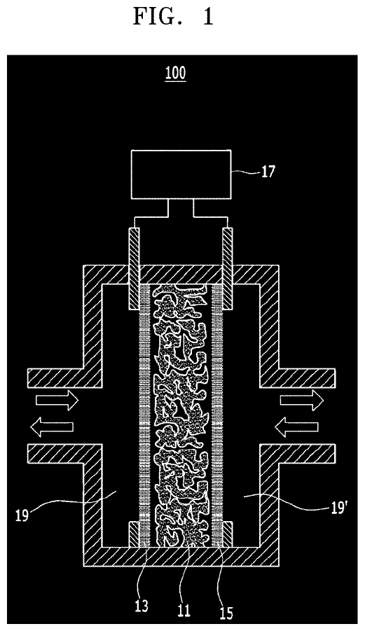

Hereinafter, an electroosmotic pump according to an embodiment of the present disclosure will be described with reference to FIGS. 1 to 3.

FIG. 1 is a configuration diagram schematically showing an electroosmotic pump according to an embodiment of the present disclosure.

Referring to FIG. 1, an electroosmotic pump 100 includes a membrane 11; a first electrode 13, which is disposed on a surface of the membrane 11 and includes a first porous support including a first conductor and a first precious metal and a first conductive polymer positioned in or on at least some of pores and surfaces of the first porous support; and a second electrode 15, which is disposed on another surface of the membrane 11 and includes a second porous support including a second conductor and a second precious metal and a second conductive polymer positioned in or on at least some of pores and surfaces of the second porous support.

As the first electrode (13) includes the first precious metal and the first conductive polymer and the second electrode (15) includes the second precious metal and the second conductive polymer, oxidation and reduction mainly occur at the first precious metal and the second precious metal during a low-voltage driving of the electroosmotic pump 100 and oxidation and reduction mainly occur at the first conductive polymer and the second conductive polymer during a high-voltage driving of the electroosmotic pump 100?). Therefore, the electroosmotic pump 100 may be driven in a wide voltage range while minimizing generation of a gas. Also, since an improved current density may be provided at the same voltage, thereby effectively improving the efficiency of driving). Also, corrosion of electrodes may be prevented, thereby effectively improving the stability of the electrodes.

For example, when a precious metal is platinum and a conductive polymer is poly(3,4-ethylene dioxythiophene):poly(styrene sulfonate)[PEDOT:PSS], reactions at electrodes are as follows.

<Formula 1> Reaction at Precious Metal

anode(+) H₂O→1/2O₂(g)+2H^{+ +}2e⁻

cathode(−) 2H₂O+2e⁻→H₂(g)+2OH⁻

<Formula 2> Reaction at Conductive Polymer

PEDOT⁰+M⁺:PSS⁻↔PEDOT⁺:PSS⁻+M⁺+e⁻

The first electrode 13 and the second electrode 15 are connected to a power supply unit 17. The first electrode 13 and the second electrode 15 may be connected to the power supply unit 17 through, for example, a lead wire, but as long as the first electrode 13 and the second electrode 15 may be electrically connected to the power supply unit 17, the connection means therebetween is not limited to the lead wire.

The membrane 11 is installed in fluid paths 19 and 19′ through which a fluid moves and may have a porous structure to enable movements of the fluid and ions.

The membrane 11 may include at least one selected from the group consisting of porous ceramics, anionic organic polymers, cationic organic polymers, and combinations thereof, each of which does not exhibit conductivity, but is not limited thereto.

The membrane 11 may be, for example, a frit-type membrane prepared by thermally calcining spherical silica. However, the present disclosure is not limited thereto, and any material capable of inducing an electrokinetic phenomenon due to zeta potential, such as porous silica, porous alumina, etc., may be used.

The spherical silica used to form the membrane 11 may have a diameter from about 20 nm to about 1 μm. Specifically, the spherical silica may have a diameter from about 50 nm to about 700 nm. More specifically, the spherical silica may have a diameter from about 100 nm to about 500 nm. When the diameter of the spherical silica is within the above-stated ranges, the electroosmotic pump 100 may generate a higher pressure.

The membrane 11 may have a thickness from about 20 μm to about 10 mm. Specifically, the membrane 11 may have a thickness from about 300 μm to about 5 mm. More specifically, the membrane 11 may have a thickness from about 500 μm to about 2 mm. When the thickness of the membrane 11 is within the above-stated ranges, the membrane 11 may exhibit sufficient strength to withstand the mechanical shock applied during manufacturing, use, and storage of the electroosmotic pump 100, and the electroosmotic pump 100 may exhibit a sufficient flow rate to be used as a drug delivery pump.

The first electrode 13 may include the first porous support including the first conductor and the first precious metal and the first conductive polymer positioned in or on at least some of pores and surfaces of the first porous support. In detail, the first precious metal and the first conductive polymer may be present in a form that covers all surfaces of the first porous support or all of the pores and all of the surfaces of the first porous support.

The second electrode 15 may include the second porous support including the second conductor and the second precious metal and the second conductive polymer positioned in or on at least some of pores and surfaces of the second porous support. In detail, the second precious metal and the second conductive polymer may be present in a form that covers all surfaces of the second porous support or all of the pores and all of the surfaces of the second porous support.

The first conductor and the second conductor may be the same as or different from each other.

The first porous support and the second porous support may be the same as or different from each other.

The first precious metal and the second precious metal may be the same as or different from each other.

The first conductive polymer and the second conductive polymer may be the same as or different from each other.

Since the first electrode 13 and the second electrode 15 have a porous structure, fluids and ions may be effectively moved.

The first conductor and the second conductor may be the same as or different from each other and may each independently include at least one selected from the group consisting of carbon, precious metals, conductive metals, conductive polymers, and combinations thereof. However, the present disclosure is not limited thereto.

As described above, when a porous support including a conductor is used for the first electrode 13 and the second electrode 15, ions may be effectively generated and fluids may be smoothly transferred. When a porous support including a conductor is used for an electrode, the specific surface area of the electrode may be increased, and thus the area of an electrode capable of participating in an electrochemical reaction may be increased.

The first porous support and the second porous support may be the same as or different from each other and may each independently have the shape of a non-woven fabric, a fabric, a sponge, or a combination thereof. However, the shape of the first porous support and the second porous support is not limited thereto, as long as the first porous support and the second porous support have porosity to enable movements of fluids and ions.

The first porous support and the second porous support may be the same as or different from each other and may each independently have a pore size from about 0.1 μm to about 500 μm. Specifically, the first porous support and the second porous support may have a pore size from about 1 μm to about 400 μm. More specifically, the first porous support and the second porous support may have a pore size from about 5 μm to about 300 μm. Even more specifically, the first porous support and the second porous support may have a pore size from about 10 μm to about 200 μm. When the pore sizes of the first porous support and the second porous support are within the above-stated ranges, fluids and ions may effectively move, thereby effectively improving the stability, lifespan characteristics, and the efficiency of the electroosmotic pump 100.

The first porous support and the second porous support may be the same as or different from each other and each independently have a porosity from about 5% to about 95%. Specifically, the first porous support and the second porous support may have a porosity from about 40% to about 80%. More specifically, the first porous support and the second porous support may have a porosity from about 50% to about 70%. When the porosity of the first porous support and the second porous support are within the above-stated ranges, fluids and ions may effectively move, thereby effectively improving the stability, lifespan characteristics, and the efficiency of the electroosmotic pump 100.

As the first precious metal and the second precious metal, any material capable of inducing a pair of reactions in which an oxidizing electrode and a reducing electrode exchange cations, e.g., hydrogen icons (H+), and forming a reversible electrochemical reaction may be used.

In detail, the first precious metal and the second precious metal may be the same as or different from each other and may each independently include one or more selected from the group consisting of platinum, gold, silver, palladium, iridium, and combinations thereof.

In the case of using a precious metal as described above, the stability, the lifespan characteristics, and the efficiency of the electroosmotic pump 100 may be effectively improved by suppressing gas generation during oxidation and reduction reactions.

The first precious metal and the second precious metal may be the same as or different from each other and each independently have an average particle diameter from about 1 nm to about 1 μm. Specifically, the first precious metal and the second precious metal may each have an average particle diameter from about 3 nm to about 500 nm. More specifically, the first precious metal and the second precious metal may each have an average particle diameter from about 5 nm to about 300 nm. Even more specifically, the first precious metal and the second precious metal may each have an average particle diameter from about 10 nm to about 100 nm. When the average particle diameter of the first precious metal or the second precious metal is within the above-stated ranges, the driving voltage of the electroosmotic pump 100 may be lowered, and the precious metal and a conductive polymer may effectively complement each other in in oxidation and reduction reactions. Therefore, the efficiency of the electroosmotic pump 100 may be effectively improved. By effectively controlling the size of precious metal nanoparticles, the degree of dispersion of the precious metal nanoparticles may be controlled in a dispersed liquid, thereby appropriately controlling the ratio of organic substances and inorganic substances that may participate in an even electrochemical reaction.

The first precious metal and the second precious metal may be the same as or different from each other and may each independently be and selectively adsorbed or impregnated on a carbon catalyst. When a precious metal is adsorbed or supported on a carbon catalyst, the precious metal may be effectively dispersed in the process of manufacturing an electrode using the same, and stability of manufacturing may be ensured even in the drying process.

The carbon catalyst is a material containing carbon and may have a smooth surface or an uneven surface or may have a porous structure. However, the present disclosure is not limited thereto, and any catalyst capable of absorbing or supporting a precious metal may be unlimitedly used.

The carbon catalyst may have an average particle diameter from about 10 nm to about 5 μm. Specifically, the carbon catalyst may have an average particle diameter from about 50 nm to about 2 μm. More specifically, the carbon catalyst may have an average particle diameter from about 100 nm to about 1 μm. When the average particle diameter of the carbon catalyst is within the above-stated ranges, the carbon catalyst may be effectively dispersed in the process of manufacturing an electrode, and the specific surface area of the electrode may be effectively controlled.

The precious metal may be physically or chemically adsorbed or impregnated inside of the carbon catalyst or all of a portion of surfaces of the carbon catalyst. However, the present disclosure is not limited thereto.

The first precious metal and the second precious metal may be the same as or different from each other and each be independently formed through electrodeposition or coating on at least some of pores and surfaces of the porous support including a conductor by using a method like dip coating, spray coating, electroless plating, plating, vacuum deposition, coating, sol-gel process, etc. However, the present disclosure is not limited thereto, and the precious metal may be formed in or on at least some of pores and surfaces of the porous support including a conductor by using an appropriate method according to the type of the precious metal.

As the first conductive polymer and the second conductive polymer, any material capable of inducing a pair of reactions in which an oxidizing electrode and a reducing electrode exchange cations, e.g., hydrogen icons (H+), and forming a reversible electrochemical reaction may be used.

In detail, the first conductive polymer and the second conductive polymer may be the same as or different from each other and each independently include one or more selected from the group consisting of poly(3,4-ethylene dioxythiophene): poly (styrene sulfonate) [PEDOT: PSS], poly(aniline):poly(styrene sulfonate), poly(aniline):camphorsulfonic acid (PANI:CSA), poly(thiophene):poly(styrene sulfonate), polyaniline, polypyrrole, polythiophene, polythionine, quinone-based polymer, and combinations thereof.

In the case of using a conductive polymer as described above, it is possible to effectively improve the stability, the lifespan characteristics, and the efficiency of the electroosmotic pump 100 by suppressing gas generation during oxidation and reduction reactions and enabling normal operation even when some gas is generated.

The first conductive polymer and the second conductive polymer may be the same as or different from each other and each independently have an average particle diameter from about 1 nm to about 1 μm. Specifically, the first conductive polymer and the second conductive polymer may each have an average particle diameter from about 5 nm to about 500 nm. Even more specifically, the first conductive polymer and the second conductive polymer may each have an average particle diameter from about 10 nm to about 300 nm. When the average particle diameter of the first conductive polymer or the second conductive polymer is within the above-stated ranges, the conductive polymer may be evenly electrodeposited or coated on at least some of the pores and surfaces of the porous support, all of the surfaces of the porous support, or all of the pores and the surfaces of the porous support.

The first conductive polymer and the second conductive polymer may be the same as or different from each other and each be independently formed through electrodeposition or coating on at least some of pores and surfaces of the porous support including a conductor by using a method like dip coating, spray coating, electroless plating, plating, vacuum deposition, coating, sol-gel process, etc. However, the present disclosure is not limited thereto, and the conductive polymer may be formed in or on at least some of pores and surfaces of the porous support including a conductor by using an appropriate method according to the type of the conductive polymer.

The first precious metal and the first conductive polymer may be formed in pores and surfaces of the first porous support including the first conductor through electrodeposition or coating on at least some of pores and surfaces of the porous support including a conductor by using a method like dip coating, spray coating, electroless plating, plating, vacuum deposition, coating, sol-gel process, etc. However, the present disclosure is not limited thereto.

The second precious metal and the second conductive polymer may be formed in pores and surfaces of the second porous support including the second conductor through electrodeposition or coating on at least some of pores and surfaces of the porous support including a conductor by using a method like dip coating, spray coating, electroless plating, plating, vacuum deposition, coating, sol-gel process, etc. However, the present disclosure is not limited thereto.

The first electrode 13 may include the first precious metal and the first conductive polymer positioned in a weight ratio from about 1:99 to about 30:70. Specifically, the first electrode 13 may include the first precious metal and the first conductive polymer positioned in a weight ratio from about 5:95 to about 10:90. More specifically, the first electrode 13 may include the first precious metal and the first conductive polymer positioned in a weight ratio of about 10:90. The second electrode 15 may include the second precious metal and the second conductive polymer positioned in a weight ratio from about 1:99 to about 30:70. Specifically, the second electrode 15 may include the second precious metal and the second conductive polymer positioned in a weight ratio from about 5:95 to about 10:90. More specifically, the second electrode 15 may include the second precious metal and the second conductive polymer positioned in a weight ratio of about 10:90. The weight ratio of the first precious metal and the first conductive polymer and the weight ratio of the second precious metal and the second conductive polymer may be the same as or different from each other. When the weight ratio of a precious metal and a conductive polymer is within the above-stated ranges, oxidation and reduction mainly occur at the precious metal during a low-voltage driving of the electroosmotic pump 100 and oxidation and reduction mainly occur at the conductive polymer during a high-voltage driving of the electroosmotic pump 100. Therefore, the electroosmotic pump 100 may be driven in a wide voltage range while minimizing generation of a gas. Also, since an improved current density may be provided at the same voltage, thereby effectively improving the efficiency of driving. Also, the specific surface area of electrodes may be effectively controlled.

The first electrode 13 may have a structure in which the first precious metal and the first conductive polymer are formed in the form of a first thin-film on at least some of the pores and the surfaces of the first porous support, on all of the surfaces of the first porous support, or all of the pores and the surfaces of the first porous support. However, the present disclosure is not limited thereto.

The second electrode 15 may have a structure in which the second precious metal and the second conductive polymer are formed in the form of a second thin-film on at least some of the pores and the surfaces of the second porous support, on all of the surfaces of the second porous support, or all of the pores and the surfaces of the second porous support. However, the present disclosure is not limited thereto.

The first thin-film and the second thin-film may be the same as or different from each other and each independently have a thickness from about 1 nm to about 2 μm. Specifically, the first thin-film and the second thin-film may each have a thickness from 10 nm to about 1 μm. More specifically, the first thin-film and the second thin-film may each have a thickness from about 50 nm to about 500 nm. When the thickness of each of the first thin-film and the second thin-film is within the above-stated ranges, an electrochemical reaction may be effectively controlled, and thus the current density may be effectively controlled.

The driving voltage of the electroosmotic pump may be from about 0.1 V to about 5 V. Specifically, the driving voltage of the electroosmotic pump may be from about 1 V to about 4 V. More specifically, the driving voltage of the electroosmotic pump may be from about 2 V to about 4 V. The electroosmotic pump 100 may be stably driven in a wide voltage range while minimizing gas generation by including a precious metal and a conductive polymer positioned in each electrode. Also, since the driving voltage may be changed in a wide range, the pressure and the flow rate of a fluid may be effectively adjusted.

The power supply unit 17 is connected to the first electrode 13 and the second electrode 15 and supply power thereto to induce electrochemical reactions, and the electrochemical reactions of the first electrode 13 and the second electrode 15 occur as cations move.

The power supply unit 17 may alternately supply voltages of opposite polarities to the first electrode 13 and the second electrode 15. At this time, alternate supplying of voltages of opposite polarities by the power supply unit 17 may include the meaning of supplying currents in opposite directions. Due to the process, the electroosmotic pump 100 may generate pressure (pumping force) through the movement of a fluid, and, at the same time, consumption and regeneration of electrochemical reaction materials of the first electrode 13 and the second electrode 15 may occur repeatedly.

For example, the power supply unit 17 may include a DC voltage supply unit (not shown) for supplying a DC voltage to each of the first electrode 13 and the second electrode 15. Also, the power supply unit 17 may include a voltage direction changing unit (not shown) that alternately switches the polarity of a DC voltage supplied to each of the first electrode 13 and the second electrode 15 at a certain time interval. Therefore, the polarity of the voltage applied to each of the first electrode 13 and the second electrode 15 may be continuously switched to an opposite polarity at a certain time interval.

The fluid paths 19 and 19′ provide movement paths in which a fluid moves around with the membrane 11, the first electrode 13, and the second electrode 15 therebetween.

Here, the fluid paths 19 and 19′ may have a container-like shape, e.g., the shape of a cylinder in which a fluid is filled. However, the shape of the fluid paths 19 and 19′ is not limited thereto.

A fluid may fill not only the fluid paths 19 and 19′, but also the membrane 11, the first electrode 13, and the second electrode 15.

The fluid paths 19 and 19′ may have openings to transmit pressure (pumping force). For example, the openings may be formed in either one or both of two spaces divided by the membrane 11, the first electrode 13, and the second electrode 15 and provide pressure (pumping force) due to movement of a fluid to the outside.

A method of manufacturing an electroosmotic pump according to another embodiment of the present disclosure includes preparing a membrane; fabricating a first electrode by forming a first precious metal and a first conductive polymer positioned in or on at least some of pores and surfaces of a first porous support including a first conductor; fabricating a second electrode by forming a second precious metal and a second conductive polymer positioned in or on at least some of pores and surfaces of a second porous support including a second conductor; and forming a membrane-electrode assembly by bringing the first electrode into contact with a surface of the membrane and brining the second electrode into contact with another surface of the membrane.

The fabricating of the first electrode may include preparing a first dispersed solution including the first precious metal, the first conductive polymer, and a first solvent and deep-coating or spray-coating the first dispersed solution on the first porous support including the first conductor. However, the present disclosure is not limited thereto.

The fabricating of the second electrode may include preparing a second dispersed solution including the second precious metal, the second conductive polymer, and a second solvent and deep-coating or spray-coating the second dispersed solution on the second porous support including the second conductor. However, the present disclosure is not limited thereto.

Here, the first precious metal may be used while being adsorbed or impregnated on a first carbon catalyst. The second precious metal may be used while being adsorbed or impregnated on a second carbon catalyst.

Descriptions of the membrane, the first conductor, the second conductor, the first porous support, the second porous support, the first precious metal, the second precious metal, the first conductive polymer, the second conductive polymer, and the carbon catalyst are identical to those already given above unless described below otherwise.

When the first electrode and the second electrode are fabricated using the first dispersed solution and the second dispersed liquid, the process of fabricating an electrode may be simplified, thereby reducing the process cost for improved economic efficiency.

Any solvent capable of dispersing the first precious metal and the first conductive polymer and any solvent capable of dispersing the second precious metal and the second conductive polymer may be used as first solvent and the second solvent, respectively. For example, the first solvent and the second solvent may be the same as or different from each other and each independently include one or more selected from the group consisting of alcohol solvents like ethanol and isopropyl alcohol, water, and combinations thereof. However, the present disclosure is not limited thereto.

The first dispersed solution and the second dispersed solution may be the same as or different from each other and each independently contain additives like a binder and a dispersion control agent as needed.

The forming of the membrane-electrode assembly may be performed by bringing the first electrode into contact with a surface of the membrane, bringing the second electrode into contact with another surface of the membrane, and pressing them. However, the present disclosure is not limited thereto.

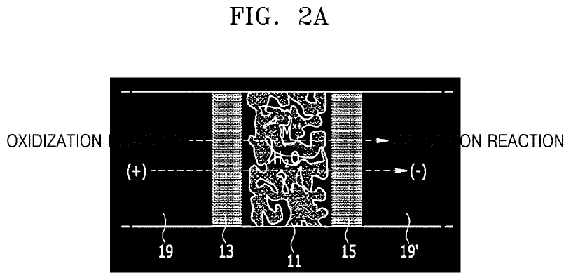

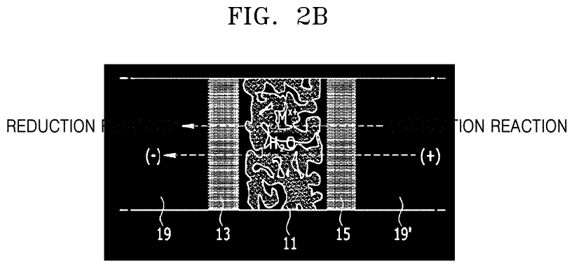

FIGS. 2A and 2B are conceptual diagrams schematically showing a reversible electrode reaction in an electroosmotic pump according to an embodiment of the present disclosure and movement of ions and a fluid resulting therefrom.

Referring to FIGS. 2A and 2B, when power is supplied with different voltages, e.g., voltages of different polarities, to the first electrode 13 and the second electrode 15 through the power supply unit 17, a voltage difference is generated between the first electrode 13 and the second electrode 15.

Due to the voltage difference, cations (M^{x+}) are generated as a result of an electrode reaction at an oxidizing electrode, and, as the generated cations (M^{x+}) move to a reducing electrode, the cations (M^{x+}) drag a fluid together, thereby generating a flow rate and a pressure (pumping force).

When power is supplied to the first electrode 13 and the second electrode 15 through the power supply unit 17, by alternately supplying voltages of different polarities, the oxidizing electrode and the reducing electrode may be switched, thereby changing moving directions of ions and a fluid, a flow rate, and a pressure (pumping force).

When an electrode served as an oxidizing electrode is switched to serve as a reducing electrode due to alternated supply of voltages of different polarities, an electrochemical reaction material consumed when the electrode is used as the oxidizing electrode may be regenerated while the electrode is being used as the reducing electrode, and vice versa. As a result, the electroosmotic pump may be continuously operated.

When the types of precious metals and conductive polymers are changed in the first electrode 13 and the second electrode 15, it is natural that the reduction reaction formula may be changed accordingly, thereby changing cations to be generated and moved.

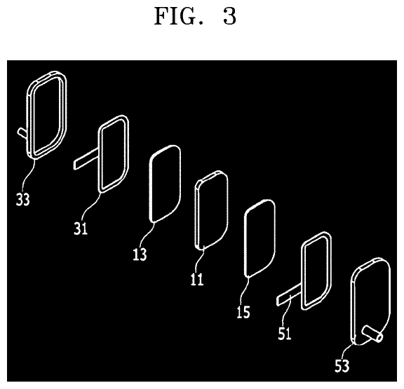

FIG. 3 is an exploded perspective view of an electroosmotic pump according to an embodiment of the present disclosure.

Referring to FIG. 3, the membrane 11 may have a rectangular shape with rounded corners, but the present disclosure is not limited thereto. Here, a coating material, a blocking sheet, an adhesive sheet, etc. may be bonded to the outer peripheral surface of the membrane 11 to prevent the leakage of a fluid.

Also, the first electrode 13 and the second electrode 15 may each have a rectangular shape with rounded corners to correspond to the shape of the membrane 11, and, in this case, a coating material, a blocking sheet, an adhesive sheet, etc. may also be bonded to the outer peripheral surfaces of the first electrode 13 and the second electrode 15 to prevent the leakage of a fluid.

A first fluid path 19 (refer to FIG. 1) may include a hollow first cap 33 adhered to the first electrode 13.

Also, a second fluid path 19′ (refer to FIG. 1) may include a hollow second cap 53 adhered to the second electrode 15.

The electroosmotic pump 100 may include a first contact strip 31 fitted to an outer circumferential surface of the first electrode 13.

Also, the electroosmotic pump 100 may include a second contact strip 51 fitted to an outer circumferential surface of the second electrode 15.

The first contact strip 31 and the second contact strip 51 may be connected to the power supply unit 17 to transmit voltages or currents to the first electrode 13 and the second electrode 15, respectively.

The first contact strip 31 and the second contact strip 51 may each include a conductive material. In detail, the first contact strip 31 and the second contact strip 51 may each include silver (Ag), copper (Cu), or the like, but the present disclosure is not limited thereto.

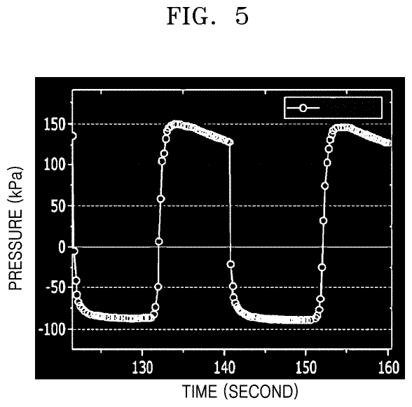

As shown in FIG. 5, the first contact strip 31 and the second contact strip 51 may have a ring-like shape to be fitted to the outer circumferential surfaces of the first electrode 13 and the second electrode 15. However, the present disclosure is not limited thereto.

According to another embodiment of the present disclosure, a fluid pumping system including an electroosmotic pump is provided. Since the fluid pumping system may be formed to have a structure commonly used in the art, detailed descriptions thereof will be omitted.

Hereinafter, the present disclosure will be described in more detail through embodiments and comparative examples. However, the embodiments and the comparative examples below are for illustrative purposes and are not intended to limit the present disclosure.

### EXAMPLES

### Example 1: Preparation of Electrode

An A5 size sheet of porous carbon paper (JNTG, GDL grade) having a thickness of about 0.26 mm was treated with oxygen plasma for about 30 minutes to make it hydrophilic. The hydrophilic porous carbon paper was placed in an electropolymerization tank containing a poly(aniline):poly(styrene sulfonate) electropolymerization solution [prepared by mixing polyaniline, poly(4-styrene sulfonic acid){poly(4-stylenesulfonic acid)} with water and heating the mixture at about 80° C.], a current was set to about 3 A as compared to a reference electrode, and electropolymerization was performed for about 30 minutes. The porous carbon paper on which polyaniline electropolymerization has been completed was taken out, washed with ultrapure water, and dried.

After the porous carbon paper on which poly(aniline):poly(styrene sulfonate) electropolymerization has been completed was fixed to a support designed for spray application, an organic-inorganic hybrid coating solution [a solution prepared by mixing small amounts of diethylene glycol binder and sodium dodecyl sulfate dispersion control agent with an aqueous dispersed solution of a carbon catalyst supporting poly(3,4-ethylene dioxythiophene):poly(styrene sulfonate) and platinum nanoparticles] was evenly applied onto the porous carbon paper by using a spray device having a nozzle, the porous carbon paper was put into an oven while being fixed to the support, and was dried.

After drying, the carbon catalyst supporting poly(3,4-ethylene dioxythiophene):poly(styrene sulfonate) and platinum nanoparticles was formed on the porous carbon paper to a thickness of about 500 nm.

The electrode prepared as described above was used as an electrode for an electroosmotic pump.

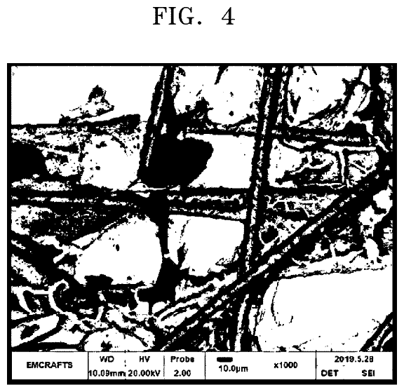

FIG. 4 is an electron micrographic image of an electrode prepared according to Example 1. FIG. 4 shows that electropolymerized poly(aniline):poly(styrene sulfonate) and the carbon catalyst supporting poly(3,4-ethylene dioxythiophene):poly(styrene sulfonate) and platinum nanoparticles are applied on the porous carbon paper, and it may be confirmed that the small grains seen in the electron micrograph are carbon catalysts supporting platinum nanoparticles.

Example 2: Manufacturing of Electroosmotic Pump

An electroosmotic pump was manufactured by positioning the electrode and membrane in the same manner as in FIG. 3. An electrode fabricated according to Example 1 was used as the electrode. The membrane was processed into a rectangular shape using spherical silica having a diameter of 300 nm through a molding, press-molding, and sintering and was staked with an electrode.

To apply a voltage from the outside to the inside of the electroosmotic pump, a suspension strip was placed. To measure a pressure/a flow rate due to the movement of a fluid during the operation of the electroosmotic pump, a plastic housing with water transfer openings was placed on both sides of the electroosmotic pump and was sealed by using an epoxy resin.

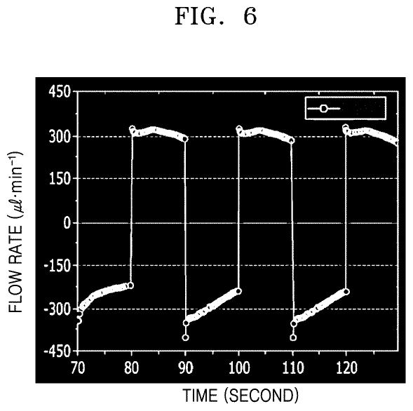

FIG. 5 is a graph of pressures generated by an electroosmotic pump manufactured according to Example 2, and FIG. 6 is a graph of flow rates of the electroosmotic pump manufactured according to Example 2. Pressures and flow rates generated when voltages of 4 V and −4 V are alternately applied every 10 seconds to a first electrode and a second electrode with a porous membrane provided therebetween were measured by using a fluid pressure sensor and a fluid flow rate sensor. The maximum pressure at a portion that drags a fluid based on the position of the fluid pressure sensor cannot exceed −100 kPa due to a physical limit.

From results shown in FIGS. 5 and 6, it may be observed that, in the electroosmotic pump manufactured according to Example 2, a fluid moves rapidly even when the electroosmotic pump is driven with 4 V, and thus the pressure of the electroosmotic pump is rapidly formed.

However, an organic-inorganic hybrid electrode according to an embodiment of the present disclosure may secure the driving stability of an electroosmotic pump while minimizing the contact area of an inorganic metal capable of inducing such electrolysis.

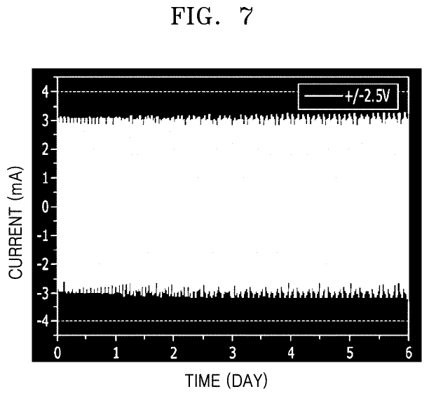

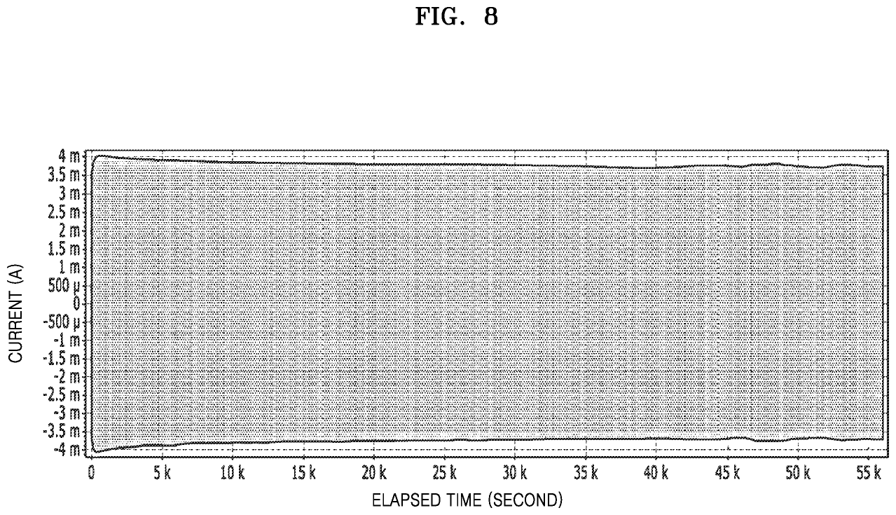

FIG. 7 is a graph of current characteristics obtained when an electroosmotic pump manufactured according to Example 2 was alternately driven at 2.5 V and −2.5 V every 5 seconds for 4 days or longer, and FIG. 8 is a graph of current characteristics obtained when an electroosmotic pump manufactured according to Example 2 was alternately driven at 4 V and −4 V every 5 seconds for 5500 cycles or more.

From results shown in FIGS. 7 and 8, it may be confirmed that the electroosmotic pump manufactured according to Example 2 may secure long-term driving stability that cannot be achieved by a conventional electroosmotic pump manufactured using only metals like platinum.

### INDUSTRIAL APPLICABILITY

Although exemplary embodiments of the present disclosure have been described above, the present disclosure is not limited thereto, and various modifications may be made within the scope of the claims, the detailed description of the disclosure, and the accompanying drawings, and the modifications also fall within the scope of the disclosure.

1. An electroosmotic pump comprising:
a membrane;
an electrode A provided on a surface of the membrane; and
an electrode B provided on another surface of the membrane,
wherein one or both of the electrode A and the electrode B comprises a porous support comprising a conductor and a precious metal and a conductive polymer positioned in or on at least some of pores and surfaces of the porous support.

2. An electroosmotic pump comprising:
a membrane;
a first electrode disposed on a surface of the membrane and comprising a first porous support including a first conductor and a first precious metal and a first conductive polymer positioned in or on at least some of pores and surfaces of the first porous support; and
a second electrode disposed on another surface of the membrane and comprising a second porous support including a second conductor and a second precious metal and a second conductive polymer positioned in or on at least some of pores and surfaces of the second porous support.

3. The electroosmotic pump of claim 2, wherein the membrane comprises one or more selected from the group consisting of porous ceramics, anionic organic polymers, cationic organic polymers, and combinations thereof, each of which does not exhibit conductivity.

4. The electroosmotic pump of claim 2, wherein the first conductor and the second conductor are the same as or different from each other and each independently comprises at least one selected from the group consisting of carbon, precious metals, conductive metals, conductive polymers, and combinations thereof.

5. The electroosmotic pump of claim 2, wherein the first porous support and the second porous support are the same as or different from each other and each independently have a pore size from about 0.1 μm to about 500 μm.

6. The electroosmotic pump of claim 2, wherein the first porous support and the second porous support are the same as or different from each other and each independently have a porosity from about 5% to about 95%.

7. The electroosmotic pump of claim 2, wherein the first precious metal and the second precious metal are the same as or different from each other and each independently include one or more selected from the group consisting of platinum, gold, silver, palladium, iridium, and combinations thereof.

8. The electroosmotic pump of claim 2, wherein the first precious metal and the second precious metal are the same as or different from each other and each independently have an average particle diameter from about 1 nm to about 1 μm.

9. The electroosmotic pump of claim 2, wherein the first precious metal and the second precious metal are the same as or different from each other and are each independently adsorbed or impregnated on a carbon catalyst.

10. The electroosmotic pump of claim 2, wherein the first conductive polymer and the second conductive polymer are the same as or different from each other and each independently comprise one or more selected from the group consisting of poly(3,4-ethylene dioxythiophene): poly (styrene sulfonate) [PEDOT: PSS], poly(aniline):poly(styrene sulfonate), poly(aniline):camphorsulfonic acid (PANI:CSA), poly(thiophene):poly(styrene sulfonate), polyaniline, polypyrrole, polythiophene, polythionine, quinone-based polymer, and combinations thereof.

11. The electroosmotic pump of claim 2, wherein the first conductive polymer and the second conductive polymer are the same as or different from each other and each independently have an average particle diameter from about 1 nm to about 1 μm.

12. The electroosmotic pump of claim 2, wherein a weight ratio of the first precious metal and the first conductive polymer is from 1:99 to 30:70, a weight ratio of the second precious metal and the second conductive polymer is from 1:99 to 30:70, and the weight ratio of the first precious metal and the first conductive polymer and the weight ratio of the second precious metal and the second conductive polymer are the same as or different from each other.

13. The electroosmotic pump of claim 2, wherein the first precious metal and the first conductive polymer are formed in a form of a first
thin-film on at least some of the pores and the surfaces of the first porous support, and the second precious metal and the second conductive polymer are formed in a form of a second thin-film on at least some of the pores and the surfaces of the second porous support.

14. The electroosmotic pump of claim 13, wherein the first thin-film and the second thin-film are the same as or different from each other and each independently have a thickness from about 1 nm to about 2 μm.

15. The electroosmotic pump of claim 1, wherein a driving voltage of the electroosmotic pump is from about 0.1 V to about 5 V.

16. The electroosmotic pump of claim 2, wherein a driving voltage of the electroosmotic pump is from about 0.1 V to about 5 V.

17. A method of manufacturing an electroosmotic pump, the method comprising:
preparing a membrane;
preparing a first electrode by forming a first precious metal and a first conductive polymer positioned in or on at least some of pores and surfaces of a first porous support comprising a first conductor;
preparing a second electrode by forming a second precious metal and a second conductive polymer positioned in or on at least some of pores and surfaces of a second porous support comprising a second conductor; and
forming a membrane-electrode assembly by bringing the first electrode in contact with a surface of the membrane and bringing the second electrode in contact with another surface of the membrane.

18. The method of claim 17, wherein the preparing of the first electrode comprises preparing a first dispersed solution including the first precious metal, the first conductive polymer, and a first solvent and deep-coating or spray-coating the first dispersed solution on the first porous support comprising the first conductor.

19. The method of claim 17, wherein the preparing of the second electrode comprises preparing a second dispersed solution including the second precious metal, the second conductive polymer, and a second solvent and deep-coating or spray-coating the second dispersed solution on the second porous support comprising the second conductor.

20. A fluid pumping system comprising the electroosmotic pump according to claim 1.
